(19) United States
(12) Patent Application Publication
(10) Pub. No.: US 2019/0069847 A1
(43) Pub. Date: Mar. 7, 2019

(54) MODULAR FINGER CUFF

(71) Applicant: Edwards Lifesciences Corporation, Irvine, CA (US)

(72) Inventors: Olaf Schraa, Amsterdam (NL); Peiyuan Li, Amsterdam (NL)

(21) Appl. No.: 16/117,071

(22) Filed: Aug. 30, 2018

Related U.S. Application Data
(60) Provisional application No. 62/555,425, filed on Sep. 7, 2017.

(51) Int. Cl. A61B 5/00; A61B 5/021

(52) U.S. Cl. CPC A61B 5/6826; A61B 5/02141; A61B 5/02427; A61B 5/02108; A61B 5/6843

(57) ABSTRACT

Disclosed is a finger cuff that is attachable to a patient's finger to be used in measuring the patient's blood pressure by a blood pressure measurement system. The finger cuff includes a plethysmograph that is detachable from the finger cuff. The plethysmograph includes a light emitting diode (LED)-photodiode (PD) pair that aids in measuring the patient's blood pressure by the blood pressure measurement system. The finger cuff further includes a bladder mountable within the finger cuff. The bladder includes a plethysmograph portion such that the plethysmograph is effectively positioned against the plethysmograph portion of the bladder when the finger cuff is attached to the patient's finger.

### CROSS-REFERENCE TO RELATED APPLICATIONS

This application claims the benefit of priority to U.S. Provisional Application No. 62/555,425, filed Sep. 7, 2017, which is incorporated herein by reference.

### BACKGROUND

### Field

Embodiments of the invention relate generally to non-invasive blood pressure measurement. More particularly, embodiments of the invention relate to a finger cuff for blood pressure measurement.

### Relevant Background

Volume clamping is a technique for non-invasively measuring blood pressure in which an external pressure is applied to a patient's finger in such a manner that arterial pressure may be balanced by a time varying pressure to maintain a constant arterial volume. In a properly fitted and calibrated system, the applied time varying pressure is equal to the arterial blood pressure in the finger. The applied time varying pressure may be measured to provide a reading of the patient's arterial blood pressure.

This may be accomplished by a finger cuff that is arranged around a finger of a patient. The finger cuff may include an infrared light source, an infrared sensor, and an inflatable bladder. The infrared light may be sent through the finger in which a finger artery is present. The infrared sensor picks up the infrared light and the amount of infrared light registered by the sensor may be inversely proportional to the artery diameter and indicative of the pressure in the artery.

In the finger cuff implementation, by inflating the bladder of the finger cuff, a pressure is exerted on the finger artery. If the pressure is high enough, it will compress the artery and the amount of light registered by the sensor will increase. The amount of pressure necessary in the inflatable bladder to compress the artery is dependent on the blood pressure. By controlling the pressure of the inflatable bladder such that the diameter of the finger artery is kept constant, the blood pressure may be monitored in very precise detail as the pressure in the inflatable bladder is directly linked to the blood pressure. In a typical present day finger cuff implementation, a volume clamp system is used with the finger cuff. The volume clamp system typically includes a pressure generating system and a regulating system that includes: a pump, a valve, and a pressure sensor in a closed loop feedback system that are used in the measurement of the arterial volume. To accurately measure blood pressure, the feedback loop provides sufficient pressure generating and releasing capabilities to match the pressure oscillations of the patient's blood pressure.

Unfortunately, a finger cuff is currently disposed of in its entirety once it expires (e.g., after being used on a patient). This is wasteful as an entire finger cuff may be expensive to manufacture. Accordingly, it would be beneficial to recycle and reuse some portions of an expired finger cuff to minimize material waste and reduce manufacturing costs.

### SUMMARY

Embodiments of the invention may relate to a finger cuff that is applied on a patient's finger to be used in measuring the patient's blood pressure by a blood pressure measurement system. The finger cuff includes a plethysmograph that is detachable from the finger cuff. The plethysmograph includes a light emitting diode (LED)-photodiode (PD) pair that aids in measuring the patient's blood pressure by the blood pressure measurement system. The finger cuff further includes a bladder mountable within the finger cuff. The bladder includes a plethysmograph portion such that the plethysmograph is effectively positioned against the plethysmograph portion of the bladder when the finger cuff is attached to the patient's finger.

### BRIEF DESCRIPTION OF THE DRAWINGS

FIG. 1 is a diagram of an example of a blood pressure measurement system according to one embodiment.

FIG. 2 is a diagram illustrating an example of a finger cuff according to one embodiment.

FIG. 3 is a block diagram illustrating an example environment in which embodiments of the invention may be practiced.

### DETAILED DESCRIPTION

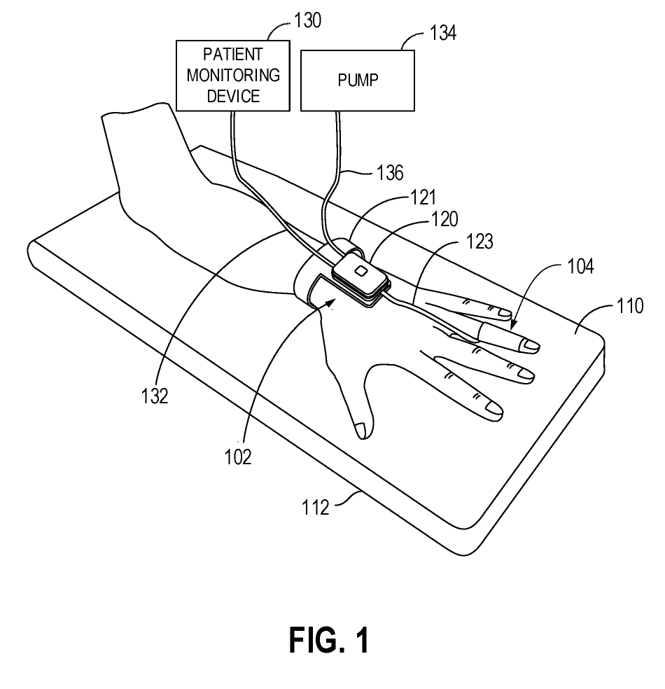

With reference to FIG. 1, which illustrates an example of a blood pressure measurement system according to one embodiment, a blood pressure measurement system 102 that includes a finger cuff 104 that may be applied to a patient's finger and a blood pressure measurement controller 120, which may be attached to the patient's body (e.g., a patient's wrist or hand) is shown.

The blood pressure measurement system 102 may further be connected to a patient monitoring device 130, and, in some embodiments, a pump 134. Further, finger cuff 104 may include a bladder (not shown) and an LED-PD pair (not shown), which are conventional for finger cuffs.

In one embodiment, the blood pressure measurement system 102 may include a pressure measurement controller 120 that includes: a small internal pump, a small internal valve, a pressure sensor, and control circuitry. In this embodiment, the control circuitry may be configured to: control the pneumatic pressure applied by the internal pump to the bladder of the finger cuff 104 to replicate the patient's blood pressure based upon measuring the plethysmograph signal received from the LED-PD pair of the finger cuff 104. Further, the control circuitry may be configured to: control the opening of the internal valve to release pneumatic pressure from the bladder; or the internal valve may simply be an orifice that is not controlled. Additionally, the control circuitry may be configured to: measure the patient's blood pressure by monitoring the pressure of the bladder based upon the input from a pressure sensor, which should be the same as patient's blood pressure, and may display the patient's blood pressure on the patient monitoring device 130.

In another embodiment, a conventional pressure generating and regulating system may be utilized, in which, a pump 134 is located remotely from the body of the patient. In this embodiment, the blood pressure measurement controller 120 receives pneumatic pressure from remote pump 134 through tube 136 and passes on the pneumatic pressure through tube 123 to the bladder of finger cuff 104. Blood pressure measurement device controller 120 may also control the pneumatic pressure (e.g., utilizing a controllable valve) applied to the finger cuff 104 as well as other functions. In this example, the pneumatic pressure applied by the pump 134 to the bladder of finger cuff 104 to replicate the patient's blood pressure based upon measuring the plethysmograph signal received from the LED-PD pair of the finger cuff 104 and measuring the patient's blood pressure by monitoring the pressure of the bladder may be controlled by the blood pressure measurement controller 120 and/or a remote computing device and/or the pump 134 and/or the patient monitoring device 130 to implement the volume clamping method. In some embodiments, a blood pressure measurement controller 120 is not used at all and there is simply a connection from tube 136 from a remote pump 134 including a remote pressure regulatory system to finger cuff 104, and all processing for the pressure generating and regulatory system, data processing, and display is performed by a remote computing device.

Continuing with this example, as shown in FIG. 1, a patient's hand may be placed on the face 110 of an arm rest 112 for measuring a patient's blood pressure with the blood pressure measurement system 102. The blood pressure measurement controller 120 of the blood pressure measurement system 102 may be coupled to a bladder of the finger cuff 104 in order to provide pneumatic pressure to the bladder for use in blood pressure measurement. Blood pressure measurement controller 120 may be coupled to the patient monitoring device 130 through a power/data cable 132. Also, in one embodiment, as previously described, in a remote implementation, blood pressure measurement controller 120 may be coupled to a remote pump 134 through tube 136 to receive pneumatic pressure for the bladder of the finger cuff 104. The patient monitoring device 130 may be any type of medical electronic device that may read, collect, process, display, etc., physiological readings/data of a patient including blood pressure, as well as any other suitable physiological patient readings. Accordingly, power/data cable 132 may transmit data to and from patient monitoring device 130 and also may provide power from the patient monitoring device 130 to the blood pressure measurement controller 120 and finger cuff 104.

As can be seen in FIG. 1, in one example, the finger cuff 104 may be attached to a patient's finger and the blood pressure measurement controller 120 may be attached on the patient's hand or wrist with an attachment bracelet 121 that wraps around the patient's wrist or hand. The attachment bracelet 121 may be metal, plastic, Velcro, etc. It should be appreciated that this is just one example of attaching a blood pressure measurement controller 120 and that any suitable way of attaching a blood pressure measurement controller to a patient's body or in close proximity to a patient's body may be utilized and that, in some embodiments, a blood pressure measurement controller 120 may not be used at all. It should further be appreciated that the finger cuff 104 may be connected to a blood pressure measurement controller described herein, or a pressure generating and regulating system of any other kind, such as a conventional pressure generating and regulating system that is located remotely from the body of the patient (e.g., a pump 134 located remotely from a patient). Any kind of pressure generating and regulating system can be used, including but not limited to the blood pressure measurement controller, and may be described simply as a pressure generating and regulating system that may be used with a finger cuff 104 including an LED-PD pair and a bladder to implement the volume clamping method.

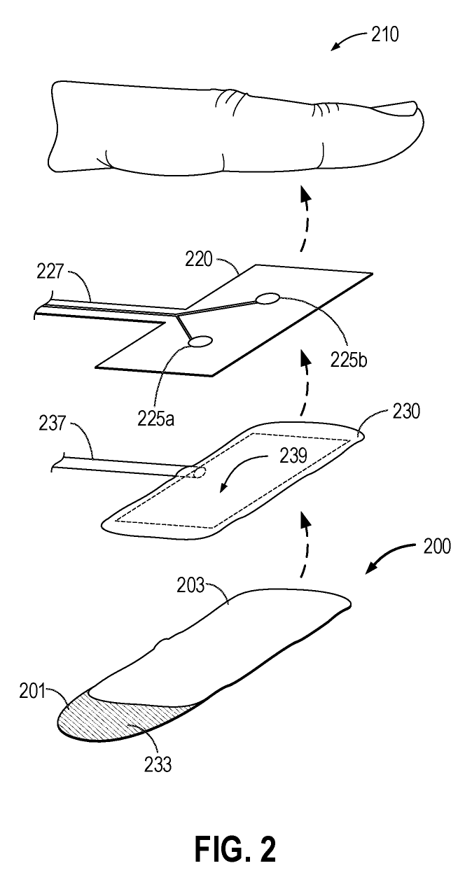

FIG. 2 is a diagram illustrating an example of a finger cuff 200 according to one embodiment. Referring to FIG. 2, finger cuff 200 may wrap around a patient's finger 210. Finger cuff 200 may include a plethysmograph 220 having an LED-PD pair 225a-b, and an inflatable bladder 230 mounted on the interior of the finger cuff 200. In one embodiment, the plethysmograph 220 may be removably attached to (e.g., using removable or reusable adhesive material) a plethysmograph portion 239 of the bladder 230 (as indicated by dotted lines). Similarly, in one embodiment, the bladder 230 may be removably attached to the interior of the finger cuff 200 (e.g., using removable or reusable adhesive material). Accordingly, it should be noted that plethysmograph 220 and/or bladder 230 may be physically separated or detached from one another and from the finger cuff 200 such that the plethysmograph 220, bladder 230, or both can be made disposable. However, in some embodiments, either the plethysmograph 220 or the bladder 230 may be permanently attached to the interior surface of the finger cuff 200.

In some embodiments, the plethysmograph 220 may be separately applied or placed (e.g., by a healthcare provider) on or around the patient's finger 210 (e.g., middle phalanx of an index, middle, or ring finger). To facilitate the placement of the plethysmograph 220 on the finger 210, an end, side, or other portion of the plethysmograph 220, on the interior, may include removable or reusable adhesive material (i.e., an adhesive layer) so that the plethysmograph 220 can be removably attached to the finger 210. It should be appreciated that this is just an example of an attachment mechanism and that any suitable type may be utilized. Subsequently, the finger cuff 200 and the bladder 230 may be effectively aligned and wrapped over the plethysmograph 220 such that the plethysmograph 220 is effectively positioned within or about the plethysmograph portion 239 of the bladder 230 and abuts against the bladder 230 when the finger cuff 200 is being attached to the patient's finger 210. Thus, in one embodiment, the plethysmograph 220 may abut against finger 210 (i.e., the patient's skin) in order to obtain the plethysmogram from the patient's finger 210. Alternatively, in an alternative embodiment, the bladder 230 may abut against finger 210 and the plethysmograph 220 may be mounted below the bladder 230.

In some embodiments, the plethysmograph 220 and bladder 230 may be of flexible material. In some embodiments, the plethysmograph 220 may be thin and of opaque, elastic material (e.g., opaque foil). The plethysmograph 220, for example, may be of a color white, black, or metallic (e.g., aluminum) so that it is opaque for infrared (IR) light. The plethysmograph 220 may be optically insulated from bladder 230 such that deformations and movements of the bladder 230 do not cause a detrimental effect on the plethysmographic (or pleth) signal generated by plethysmograph 220. For example, when utilizing a volume clamp system, the bladder 230 may be inflated and deflated to match intra-arterial pressure. Such pressure changes in the bladder 230 may influence the intensity of the plethysmogram signal, thereby distorting the measured pressure waveform (e.g., over- or underestimation of the pulse pressure). Therefore, by optically insulating the plethysmograph 220 from the bladder 230, such distortion may be minimized or entirely eliminated.

In some embodiments, the bladder 230 may be a circular bladder having a stiff exterior. In some embodiments, the LED of LED-PD pair 225a-b pair may be an organic light emitting diode (OLED).

Also, as shown in FIG. 2, the LED-PD pair 225a-b may be coupled or connected to a cable 227 through a connector (not shown), which may be attached to plethysmograph 220, to provide power to and receive data (i.e., electrical signals) from the LED-PD pair 225a-b. Additionally, the bladder 230 may be coupled or connected to a tube 237 (also through a connector, in some embodiments) to provide pneumatic pressure to the bladder 230.

Further, finger cuff 200 may include a first side 201 and a second side 203. In one embodiment, the first side 201 on the interior may include a first connecting portion 233 (e.g., a Velcro type portion) that connects with a second connecting portion, e.g., a Velcro type portion, on the exterior of the second side 203 of the finger cuff 200. In another embodiment, the first connecting portion 233 may include removable or reusable adhesive material that can be removably attached to the exterior surface of the second side 203 of the finger cuff 200. It should be appreciated that these are just some examples of an attachment mechanism and that any suitable type may be utilized.

As has been described, the plethysmograph 220 and/or bladder 230 may be physically separated or detached from one another and from the finger cuff 200 such that the plethysmograph 220, bladder 230, or both can be made disposable. Because only some of these items are disposable, the remaining portions of the finger cuff may be re-usable such that the whole finger cuff does not have to be disposed of after usage thereby minimizing material waste and reducing manufacturing costs.

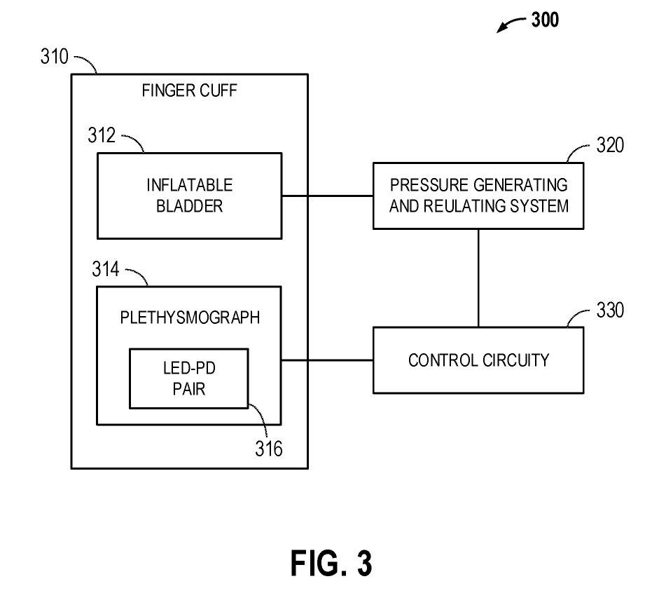

FIG. 3 is a block diagram illustrating an example environment 300 in which embodiments of the invention may be practiced. As shown, finger cuff 310 may include an inflatable bladder 312 and a plethysmograph 314. The inflatable bladder 312 may be pneumatically connected to a pressure generating and regulating system 320. The pressure generating and regulating system 320 may generate, measure, and regulate pneumatic pressure that inflates or deflates the bladder 312, and may include elements such as a pump, a valve, a sensor, control circuitry, and/or other suitable elements. When the bladder 312 is inflated, the finger cuff 310 applies a pressure to the patient's finger.

In one embodiment, the plethysmograph 314 may make continuous volumetric measurements (or plethysmogram) of arterial blood flows within the finger. In one embodiment, the plethysmograph 314 may include a LED-PD pair 316. The LED may be used to illuminate the finger skin and light absorption or reflection may be detected with the photodiode. Therefore, the plethysmogram may be generated based on the signal received from the photodiode.

The pressure generating and regulating system 320 and the plethysmograph 314 may be connected to a control circuitry 330. The control circuitry 330 may instruct the pressure generating and regulating system 320 to inflate or deflate the bladder 312 based on a pressure setting, may receive data from the plethysmograph 314, and may carry out necessary data manipulations.

It should be appreciated that aspects of the invention previously described may be implemented in conjunction with the execution of instructions by processors, circuitry, controllers, control circuitry, etc. As an example, control circuity may operate under the control of a program, algorithm, routine, or the execution of instructions to execute methods or processes in accordance with embodiments of the invention previously described. For example, such a program may be implemented in firmware or software (e.g. stored in memory and/or other locations) and may be implemented by processors, control circuitry, and/or other circuitry, these terms being utilized interchangeably. Further, it should be appreciated that the terms processor, microprocessor, circuitry, control circuitry, circuit board, controller, microcontroller, etc., refer to any type of logic or circuitry capable of executing logic, commands, instructions, software, firmware, functionality, etc., which may be utilized to execute embodiments of the invention.

The various illustrative logical blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A finger cuff attachable to a patient's finger to be used in measuring the patient's blood pressure by a blood pressure measurement system, the finger cuff comprising:
a plethysmograph including a light emitting diode (LED)-photodiode (PD) pair that aids in measuring the patient's blood pressure by the blood pressure measurement system, the plethysmograph being detachable from the finger cuff; and
a bladder mountable within the finger cuff, the bladder including a plethysmograph portion such that the plethysmograph is effectively positioned against the plethysmograph portion of the bladder when the finger cuff is attached to the patient's finger.

2. The finger cuff of claim 1, wherein the bladder is detachable from the finger cuff.

3. The finger cuff of claim 1, wherein:
the LED-PD pair is coupled to a cable that provides power to the LED-PD pair and receives electrical signals from the LED-PD pair, and
the bladder is coupled to a tube that provides pneumatic pressure to the bladder.

4. The finger cuff of claim 1, wherein the plethysmograph includes opaque and elastic material.

5. The finger cuff of claim 4, wherein an interior of the plethysmograph includes an adhesive layer that is removably attached to the patient's finger to facilitate placement of the plethysmograph on or around the patient's finger.

6. The finger cuff of claim 4, wherein the opaque and elastic material includes a white, black or metallic foil.

7. The finger cuff of claim 1, wherein the plethysmograph is optically insulated from the bladder to minimize or eliminate distortion of a pleth signal received from the LED-PD pair.

8. The finger cuff of claim 1, wherein either the bladder or the plethysmograph abuts against the patient's finger.

9. The finger cuff of claim 1, further comprising a first side and a second side, the first side including a first connecting portion and the second side including a second connecting portion, wherein the first connecting portion is removably attached to the second connecting portion to wrap the finger cuff around the patient's finger.

10. A method to measure a patient's blood pressure by a blood pressure measurement system utilizing a finger cuff, the finger cuff including a bladder mountable within the finger cuff, and a plethysmograph detachable from the finger cuff and having a light emitting diode (LED)-photodiode (PD) pair, the method comprising:
placing the plethysmograph on a patient's finger such that the LED-PD pair aids in measuring the patient's blood pressure by the blood pressure measurement system;
aligning the bladder over the plethysmograph such that the plethysmograph is effectively positioned against a plethysmograph portion of the bladder;
placing the bladder over the plethysmograph such that the bladder abuts against the plethysmograph; and
securing the finger cuff including the plethysmograph and the bladder around the patient's finger.

11. The method of claim 10, wherein the bladder is detachable from the finger cuff.

12. The method of claim 10, wherein:
the LED-PD pair is coupled to a cable that provides power to the LED-PD pair and receives electrical signals from the LED-PD pair, and
the bladder is coupled to a tube that provides pneumatic pressure to the bladder.

13. The method of claim 10, wherein the plethysmograph includes opaque and elastic material.

14. The method of claim 13, wherein an interior of the plethysmograph includes an adhesive layer that is removably attached to the patient's finger to facilitate placement of the plethysmograph on the patient's finger.

15. The method of claim 13, wherein the opaque and elastic material includes a white, black or metallic foil.

16. The method of claim 10, wherein the plethysmograph is optically insulated from the bladder to minimize or eliminate distortion of a pleth signal received from the LED-PD pair.

17. The method of claim 10, wherein either the bladder or the plethysmograph abuts against the patient's finger.

18. The method of claim 10, wherein securing the finger cuff around the patient's finger comprises:
pulling a first side of the finger cuff towards a second side of the finger cuff, and
attaching a first connecting portion of the first side to a second connecting portion of the second side, wherein the first connecting portion is removably attached to the second connecting portion to wrap the finger cuff around the patient's finger.
